(19) United States
(12) Patent Application Publication
(10) Pub. No.: US 2002/0034484 A1
(43) Pub. Date: Mar. 21, 2002

(54) Vitamin/metal salt compositions for reducing hair loss and/or promoting hair regrowth

(76) Inventors: Olivier de Lacharriere, Paris (FR); Francis Pruche, Senlis (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/902,266

(22) Filed: Jul. 11, 2001

(30) Foreign Application Priority Data
Jul. 11, 2000 (FR) 00/09063

(51) Int. Cl. A61K 7/06; A61K 33/04; A61K 33/32; A61K 31/355

(52) U.S. Cl. 424/70.1; 424/702; 424/641; 514/458

(57) ABSTRACT

Cosmetic/pharmaceutical compositions for promoting hair growth and/or retarding hair loss, and/or for increasing the mean diameter of strands of hair and/or decreasing the heterogeneity thereof, and/or for increasing hair density, and/or for improving the quality and/or the appearance of a head of hair, and/or for inducing repigmentation of the hair, contain thus effective amounts of intimate admixture of vitamin A, vitamin C, vitamin E, and zinc and selenium values.

### CROSS-REFERENCE TO PRIORITY APPLICATION

This application claims priority under 35 U.S.C. § 119 of FR-00/09063, filed Jul. 11, 2000, hereby expressly incorporated by reference.

### BACKGROUND OF THE INVENTION

1. Technical Field of the Invention

The present invention relates to novel compositions based on vitamins and on inorganic/organic metal salts for promoting regrowth of the hair and/or decreasing hair loss and/or improving the quality of the hair and/or promoting repigmentation thereof.

2. Description of the Prior Art

In humans, the growth of the hair and renewal thereof are determined principally by the activity of the hair follicles. This activity is cyclical and essentially entails three phases, namely, the anagenic phase, the catagenic phase and the telogenic phase.

The active anagenic phase, or growth phase, which is several years in duration and during which the strands of hair grow longer, is followed by a very short and transient catagenic phase which is several weeks in duration, and then by a resting or quiescent phase, termed telogenic phase, which lasts a few months.

At the end of the resting period, the hair falls out and another cycle begins. The head of hair is therefore constantly renewed and, of the approximately 150,000 hairs which a head of hair contains, at each instant, approximately 10% of the strands of hair are at rest and will therefore be replaced in a few months.

However, various factors or causes may result in temporary or permanent hair loss.

This may be loss and modification of the hair as pregnancy comes to an end (post partum) or during conditions of undernourishment or of dietary imbalance, or during conditions of aesthenia or of hormonal dysfunction, as can be the case during menopause or conclusion thereof. It may also be loss or modifications of the hair in connection with seasonal phenomena.

This may also be alopecia which is essentially due to a disturbance or disruption in hair renewal which results, initially, in the acceleration of the frequency of the cycles at the expense of the quality of the hair and then of its amount. A progressing thinning of the head of hair occurs, caused by an effect on the follicles. Regions are preferentially affected, in particular the temporal or frontal lobes in men and, in women, a diffuse alopecia of the vertex is observed.

By the term “alopecia” is thus intended the entire family of afflictions of the hair follicle, the final consequence of which is permanent, partial or general hair loss.

This, more particularly, may be androgenic alopecia. In a considerable number of cases, early hair loss occurs in genetically predisposed individuals. This, then, is androchronogenetic alopecia, which form of alopecia is especially prevalent in men.

It is known, moreover, that certain factors, such as a hormone imbalance, physiological stress, or malnutrition, can accentuate this phenomenon.

In certain dermatoses of the scalp which are inflammatory in nature, such as, for example, psoriasis or seborrhoeic dermatitis, hair loss may be greatly accentuated or promote greatly disturbed follicle cycles.

For many years, considerable research has been conducted in the cosmetics or pharmaceutical industry, to develop compositions for suppressing or reducing alopecia, and, in particular, for reducing hair loss or inducing or stimulating its growth.

In this perspective, a large number of compositions comprising very diverse active agents have already been developed, such as, for example, 2,4-diamino-6-piperidinopyrimidine 3-oxide, or “Minoxidil”, described in U.S. Pat. Nos. 4,139,619 and 4,596,812, or the many derivatives thereof, such as those described, for example, in EP 0,353,123, EP 0,356,271, EP 0,408,442, EP 0,522,964, EP 0,420,707, EP 0,459,890, EP 0,519,819.

Nonetheless, considerable need still exists to develop advantageous and useful active compounds other than those already known to this art.

In humans, it was commonly accepted for decades that administration of antioxidants played a beneficial role by decreasing the incidence of certain chronic conditions, such as cardiovascular diseases and cancers.

In particular, epidemiological studies and studies carried out in animals had bolstered the hypothesis that beta-carotene served a beneficial function in preventing the onset of certain diseases, such as those of lung cancer.

However, these results have been disputed following two randomized chemo-prevention studies, conducted in double blind versus placebo, the objective of which was to evaluate the effectiveness of supplementation with beta-carotene (alone or combined with vitamins A and E) on the incidence of lung cancers in smokers and individuals working with asbestos, and/or of cardiovascular diseases.

The first study, ATBC (Alpha-tocopherol, Beta-carotene Cancer Prevention), carried out in Finland, indicated an 18% increase in lung cancers with an additional 8% of deaths among men who received beta-carotene, out of a population of 29,139 men who were smokers.

No decrease in the incidence of lung cancers, after 5 years of administration, was found. (The effect of vitamin E and beta-carotene on the incidence of lung cancer and other cancers in male smokers. The alpha-Tocopherol, beta-Carotene Cancer Prevention Study Group, N. Engl. J. Med., 330, 1029-35 (1994)).

The second study, CARET (Beta-Carotene and Retinol Efficacy Trial), conducted in the United States (Seattle), indicated a 28% increase in lung cancers with an additional 17% of deaths, and a 26% increase in deaths due to cardiovascular diseases, in the 18,314 participants (smokers and workers exposed to asbestos), with respect to the placebo group. (G. S. Omenn, G. E. Goodman, M. D. Thorquist et al., “Effects of a combination of beta-carotene and vitamin A on lung cancer and cardiovascular disease,” N. Engl. J. Med., 334, 1150-5 (1996)).

These results confirmed and extended the unexpected results of the preventive study carried out in Finland. Specifically, far from being protective, as was implied by the epidemiological studies and those carried out in animals, administration/supplementation with beta-carotene may prove to be harmful.

In conclusion to these two trials, it has been suggested that the paradoxical effect of the increase in morbidity and in mortality observed is likely due to the properties of the beta-carotene and its capacity to generate oxidative stress. Thus, antioxidant agents can prove to be pro-oxidant and elicit deleterious effects.

Accordingly, no reason would appear to exist for including antioxidant compounds in compositions, particularly cosmetic compositions, for improving the aesthetic appearance of the head of hair, or for combating hair loss and/or promoting its regrowth.

### SUMMARY OF THE INVENTION

Surprisingly, it has now unexpectedly been determined that compositions comprising vitamins and certain metal values, e.g., inorganic/organic salts, particularly vitamins eliciting an antioxidant effect, increase the mean diameter of the strands of hair, decrease the heterogeneity of the diameters of the hairs (a symptom associated with alopecia) and increase hair density, thus reflecting regrowth.

It has also, unexpectedly, now been determined that compositions based on vitamins and on certain metal salts, particularly vitamins eliciting an antioxidant effect, induce repigmentation of the hair and thus combat whitening of the hair.

### DETAILED DESCRIPTION OF BEST MODE AND SPECIFIC/PREFERRED EMBODIMENTS OF THE INVENTION

More particularly according to the present invention, by the expression “heterogeneity of the diameters of the hairs” is intended a great variation in the diameters of the hairs over the same region of the scalp, some hairs having a physiological diameter and others having, in the immediate vicinity of these hairs, a decreased diameter (fine hairs). Thus, the term “decrease the heterogeneity of the diameters” is intended to connote an increase in the diameter of the fine hairs.

By the expression “increase hair density” is intended to increase the number of hairs per cm² of scalp.

And by the expression “improve the quality of the hair” is intended to improve the general appearance and/or the sheen and/or the ability to style the head of hair.

Thus, the present invention features compositions comprising at least a mixture of vitamin A, vitamin E, vitamin C, and zinc and selenium values, to promote regrowth of the hair and/or retard hair loss.

The present invention also features compositions comprising admixture of vitamin A, vitamin E, vitamin C, zinc and selenium, for increasing the mean diameter of the strands of hair.

This invention also features compositions comprising admixture of vitamin A, vitamin E, vitamin C, zinc and selenium, for decreasing the heterogeneity of the diameters of the strands of hair.

Too, this invention features compositions comprising admixture of vitamin A, vitamin E, vitamin C, zinc and selenium, for increasing hair density.

The present invention also features compositions comprising admixture of vitamin A, vitamin E, vitamin C, zinc and selenium, for improving the quality and/or the appearance of the head of hair.

And the present invention also features compositions comprising admixture of vitamin A, vitamin E, vitamin C, zinc and selenium, for inducing repigmentation of the hair.

The aforesaid results are more pronounced in women, and also in men experiencing the onset of alopecia.

The increase in the diameter of the strands of hair is the parameter which is most improved via the regime/regimen entailing treatment with the compositions of the invention.

The vitamins of the composition of the invention are preferably vitamins having an antioxidant effect, selected from among vitamin A (retinol or esters thereof, or its equivalent as beta-carotene), vitamin E (alpha- or gamma-tocopherol), vitamin C (ascorbic acid or salts thereof) and the B vitamins.

The compositions of the invention preferably comprise immixture of vitamin A, vitamin E and vitamin C.

The inorganic salts of the compositions of the invention are preferably selected from among the salts of zinc, selenium, iron, magnesium, copper and manganese.

Consistent herewith, the zinc is advantageously in the form of zinc gluconate, zinc oxide, zinc sulfate or zinc chloride. Similarly, the iron is advantageously in the form of ferrous fumarate, ferrous sulfate or ferrous chloride, the magnesium in the form of magnesium oxide, the copper in the form of copper oxide or copper gluconate, and the manganese in the form of manganese chloride or manganese sulfate. The selenium is advantageously in inorganic state, such as sodium selenite, or in organic state, such as selenocysteine, or in the form of selenoyeast.

The compositions of the invention preferably comprise admixture of zinc and selenium values, for example intimate admixture of zinc sulfate and sodium selenite.

The amount of each of the elements of the subject compositions depends, of course, on the desired effect, and may vary over wide limits.

According to the invention, the subject compositions advantageously comprise vitamin A in an amount ranging from 0.1 mg to 3 mg, preferably from 0.5 mg to 1.5 mg, vitamin C in an amount ranging from 50 mg to 240 mg, preferably from 100 mg to 140 mg, vitamin E in an amount ranging from 10 mg to 60 mg, preferably from 40 mg to 50 mg, zinc in an amount ranging from 10 mg to 40 mg, preferably from 15 mg to 25 mg, and selenium in an amount ranging from 40 μg to 150 μg, preferably from 70 μg to 120 μg.

When the vitamin A is in the form of beta-carotene equivalent, the composition advantageously comprises from 0.6 mg to 18 mg of beta-carotene, and preferably from 3 mg to 4.5 mg.

A very preferred composition of the invention comprises 1 mg of vitamin A (or 6 mg of beta-carotene), 120 mg of vitamin C, 30 mg of vitamin E, 20 mg of zinc and 100 μg of selenium.

The compositions of the invention may also comprise B vitamins and/or iron and/or magnesium, copper and manganese. The subject compositions may also comprise other antioxidant active agents, such as superoxide dismutases (SODs), catalases, peroxidases (for example glutathione peroxidase) and/or synthetic molecules or associations exhibiting enzymatic activities which mimic these enzymes (for example manganese or copper complexes with an SOD-like activity, such as copper diisopropylsalicylate or Mn(III) tetrakis(4-benzoic acid)porphyrin chloride).

The present invention thus features compositions based on vitamins and on inorganic/organic salts, as described above, also comprising B vitamins and/or iron and/or magnesium, copper and manganese and/or at least one other antioxidant active agent, such as an SOD, a catalase, a peroxidase (for example glutathione peroxidase) and/or a synthetic molecule or association exhibiting enzymatic activities which mimic these enzymes (for example manganese or copper complexes with an SOD-like activity, such as copper diisopropylsalicylate or Mn(III) tetrakis(4-benzoic acid)porphyrin chloride).

The subject compositions may also comprise sulfur-containing amino acids or precursors of sulfur-containing amino acids, such as, for example, cysteine, cystine, N-acetylcysteine, glutathione and/or esters thereof, or oxathiazolidine. These compositions preferably also comprise L-cystine.

The compositions of the invention may be formulated in any pharmaceutical form whatsoever which is conventionally employed for treating the scalp and which is suitable both for topical application to the hair and for oral administration, characteristically including a cosmetically/pharmaceutically acceptable medium (vehicle, diluent or carrier therefor).

Preferably, the compositions of the invention are formulated for oral administration.

The compositions of the invention can be for cosmetic or dermatological purposes. Preferably, the compositions of the invention are for cosmetic purposes.

Very preferably, the compositions of the invention are cosmetic compositions formulated for oral administration.

The compositions of the invention are considered cosmetic since they improve or enhance the general appearance of the individual user.

For oral administration, the compositions of the invention may be in any suitable form, particularly in the form of a drinkable solution, of a syrup, of a tablet, of a gelatin capsule, or of a capsule.

Preferably, the compositions of the invention are formulated into gelatin capsules.

Thus, the present invention features a regime/regimen conducted for such period of time as required to elicit the desired effect, for administering a mixture of vitamins and of inorganic/organic salts, as described above, to promote regrowth of the hair and/or decrease hair loss and/or increase the mean diameter of the hairs and/or decrease the heterogeneity of the diameters of the hairs and/or increase hair density and/or improve the general appearance of the head of hair and/or promote repigmentation of the hair.

Lastly, this invention features a cosmetic hair treatment process for improving the appearance of the head of hair and/or for promoting regrowth of the hair and/or for decreasing hair loss and/or for increasing the mean diameter of the hairs and/or for decreasing the heterogeneity of the diameters of the hairs and/or for increasing hair density and/or for promoting repigmentation of the hair, comprising topically applying at least one of the compositions described above onto the scalp and/or the hair, and then, optionally, in rinsing it with water therefrom.

In order to further illustrate the present invention and the advantages thereof, the following specific examples are given, it being understood that same are intended only as illustrative and in nowise limitative.

The Examples 1-5 to follow are of specific compositions of the invention, formulated as indicated.

### EXAMPLE 1

Soft capsules:

| | | |
|---|---|---|
| Soya bean oil | 40 | mg |
| Wheatgerm oil | 85 | mg |
| Soya bean lecithins | 25 | mg |
| Vitamin A | 1 | mg |
| Vitamin C | 120 | mg |
| Vitamin E | 30 | mg |
| Zinc | 20 | mg |
| Selenium | 100 | μg |

### EXAMPLE 2

Soft capsules:

| | | |
|---|---|---|
| Soya bean oil | 40 | mg |
| Wheatgerm oil | 85 | mg |
| Soya bean lecithins | 25 | mg |
| Beta-carotene | 6 | mg |
| Vitamin C | 120 | mg |
| Vitamin E | 30 | mg |
| Zinc | 20 | mg |
| Selenium | 100 | μg |

### EXAMPLE 3

Soft capsules:

| | | |
|---|---|---|
| Soya bean oil | 40 | mg |
| Wheatgerm oil | 85 | mg |
| Soya bean lecithins | 25 | mg |
| Vitamin A | 1 | mg |
| Vitamin C | 120 | mg |
| Vitamin E | 30 | mg |
| Zinc | 20 | mg |
| Selenium | 100 | μg |
| Manganese | 5 | mg |

### EXAMPLE 4

Anti-hair-loss lotion packaged in 10 ml vials:

| | |
|---|---|
| Vitamin A | 1 mg |
| Vitamin C | 120 mg |
| Vitamin E | 30 mg |
| Zinc | 20 mg |
| Selenium | 100 μg |

solubilized in 100 ml of a mixture of:

| | |
|---|---|
| Fructose | 0.22% |
| Glucose | 0.04% |
| Urea | 0.06% |
| NaCl | 0.50% |
| Polyethylene glycol | 0.25% |
| Lactic acid | 0.05% |
| Potassium hydroxide | qs pH = 7.5 |
| Water/ethyl alcohol, 31% by weight | qs for 100.00% |

### EXAMPLE 5

Anti-hair-loss lotion packaged in 10 ml vials:

| | |
|---|---|
| Beta-carotene | 6 mg |
| Vitamin C | 120 mg |
| Vitamin E | 30 mg |
| Zinc | 20 mg |
| Selenium | 100 μg |

solubilized in 100 ml of a mixture of:

| | |
|---|---|
| Fructose | 0.22% |
| Glucose | 0.04% |
| Urea | 0.06% |
| NaCl | 0.50% |
| Polyethylene glycol | 0.25% |
| Lactic acid | 0.05% |
| Potassium hydroxide | qs pH = 7.5 |
| Water/ethyl alcohol, 31% by weight | qs for 100.00% |

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A cosmetic/pharmaceutical composition for promoting hair regrowth and/or retarding hair loss, comprising a thus effective amount of intimate admixture of vitamin A, vitamin C, vitamin E, and zinc and selenium values.

2. A cosmetic/pharmaceutical composition for increasing the mean diameter of strands of hair, comprising a thus effective amount of intimate admixture of vitamin A, vitamin C, vitamin E, and zinc and selenium values.

3. A cosmetic/pharmaceutical composition for decreasing the heterogeneity of the diameters of strands of hair, comprising a thus effective amount of intimate admixture of vitamin A, vitamin C, vitamin E, and zinc and selenium values.

4. A cosmetic/pharmaceutical composition for increasing hair density, comprising a thus effective amount of intimate admixture of vitamin A, vitamin C, vitamin E, and zinc and selenium values.

5. A cosmetic/pharmaceutical composition for improving the quality and/or the appearance of a head of hair, comprising a thus effective amount of intimate admixture of vitamin A, vitamin C, vitamin E, and zinc and selenium values.

6. A cosmetic/pharmaceutical composition for inducing repigmentation of the hair, comprising a thus effective amount of intimate admixture of vitamin A, vitamin C, vitamin E, and zinc and selenium values.

7. The cosmetic/pharmaceutical composition as defined by any of claims 1 to 6, comprising about 0.1 mg to about 3 mg of vitamin A, about 50 mg to about 240 mg of vitamin C, about 10 mg to about 60 mg of vitamin E, about 10 mg to about 40 mg of zinc and about 40 μg to about 150 μg of selenium.

8. The cosmetic/pharmaceutical composition as defined by claim 7, comprising about 0.5 mg to about 1.5 mg of vitamin A, about 100 mg to about 140 mg of vitamin C, about 40 mg to about 50 mg of vitamin E, about 15 mg to about 25 mg of zinc, and about 70 μg to about 120 μg of selenium.

9. The cosmetic/pharmaceutical composition as defined by any of claims 1 to 6, formulated for oral administration, in a cosmetically/pharmaceutically acceptable vehicle, diluent or carrier therefor.

10. The cosmetic/pharmaceutical composition as defined by any of claims 1 to 6, formulated for topical application, in a cosmetically/pharmaceutically acceptable vehicle, diluent or carrier therefor.

11. The cosmetic/pharmaceutical composition as defined by any of claims 1 to 6, further comprising iron, magnesium, copper and/or manganese values, or combination thereof.

12. The cosmetic/pharmaceutical composition as defined by any of claims 1 to 6, further comprising an additional antioxidant, a catalase, a peroxidase, a synthetic molecule or association exhibiting enzymatic activity, a sulfur-containing amino acid, or combination thereof.

13. The cosmetic/pharmaceutical composition as defined by any claim 8, comprising about 1 mg of vitamin A, about 120 mg of vitamin C, about 30 mg of vitamin E, about 20 mg of zinc, and about 100 μg of selenium.

14. The cosmetic/pharmaceutical composition as defined by any of claims 1 to 6, the vitamin A being present as beta-carotene equivalent thereof.

15. The cosmetic/pharmaceutical composition as defined by claim 14, comprising about 0.6 mg to about 18 mg of beta-carotene.

16. The cosmetic/pharmaceutical composition as defined by any of claims 1 to 6, formulated as a drinkable solution, syrup, tablets, or capsules.

17. A regime or regimen for promoting hair growth and/or retarding hair loss, and/or for increasing the mean diameter of strands of hair and/or decreasing the heterogeneity thereof, and/or for increasing hair density, and/or for improving the quality and/or the appearance of a head of hair, and/or for inducing repigmentation of the hair, comprising administering to an individual in need of such treatment, for such period of time as required to elicit the desired effect, a thus effective amount of intimate admixture of vitamin A, vitamin C, vitamin E, and zinc and selenium values.
